(19) United States
(12) Patent Application Publication
(10) Pub. No.: US 2026/0290044 A1
(43) Pub. Date: Sep. 24, 2026

(54) INFORMATION PROCESSING DEVICE, INFORMATION PROCESSING SYSTEM, INFORMATION PROCESSING METHOD, AND STORAGE MEDIUM

(71) Applicant: HONDA MOTOR CO., LTD., Tokyo (JP)

(72) Inventors: Ryuji Iwashita, Wako-shi (JP); Yuji Yasui, Wako-shi (JP); Seungho Choi, Wako-shi (JP); Kotaro Masuda, Wako-shi (JP); Tetsuya Gomi, Tokyo (JP); Yoko Umeyama, Tokyo (JP); Taiki Iwama, Tokyo (JP); Masahiro Kimura, Wako-shi (JP); Jing Zhao, Tokyo (JP); Adil Gupta, Tokyo (JP); Ryo Takebayashi, Tokyo (JP)

(21) Appl. No.: 19/550,459

(22) Filed: Feb. 26, 2026

(30) Foreign Application Priority Data
Mar. 24, 2025 (JP) 2025-048632

(51) Int. Cl. G06V 20/59; A61B 5/00; B60Q 9/00; G06V 20/54; G06V 20/58; G06V 40/16; G06V 40/18

(52) U.S. Cl. CPC G06V 20/597; A61B 5/747; B60Q 9/008; G06V 20/54; G06V 20/58; G06V 40/166; G06V 40/18

(57) ABSTRACT

An information processing device includes an acquisition unit configured to acquire a surrounding image of a vehicle and a face image of a driver of the vehicle, a risk detection unit configured to detect a risk object in the surrounding image, a visual recognition determination unit configured to determine whether the driver visually recognizes the risk object, on the basis of information relating to a position of the risk object and information relating to the face image, an abnormality determination unit configured to determine whether a first abnormality occurs in the driver, on the basis of whether a risk avoidance action to reduce a risk degree indicating a degree of risk of collision between the vehicle and the risk object is detected after determination is made that the driver visually recognizes the risk object.

### CROSS-REFERENCE TO RELATED APPLICATION

Priority is claimed on Japanese Patent Application No. 2025-048632, filed Mar. 24, 2025, the content of which is incorporated herein by reference.

### BACKGROUND OF THE INVENTION

### Field of the Invention

The present invention relates to an information processing device, an information processing system, an information processing method, and a storage medium.

### Description of Related Art

In recent years, efforts have been actively made to provide access to a sustainable transportation system with special attention to people in vulnerable situations such as aged people, people with disabilities, and children among traffic participants. To implement this, research and development for further improving the safety or convenience of traffic through development regarding means for transportation for, in particular, aged people or people with disabilities has been focused on (for example, see Patent Document 1 described below).
[Patent Document 1] Japanese Unexamined Patent Application, First Publication No. 2023-20745

### SUMMARY OF THE INVENTION

When the driver is suffering from dementia, a driver's ability to avoid a risk object is reduced. However, the driver may be unaware of dementia. When the driver continues driving while being unaware of dementia and reduction in ability due to dementia, there is a possibility that the safety or convenience of traffic is impaired.

An aspect according to the present invention has been made in consideration of such a situation, and an object of the present invention is to provide an information processing device, an information processing system, an information processing method, and a storage medium capable of detecting dementia of a driver. The present invention, in turn, contributes to development of a sustainable transportation system.

To solve the above-described problem, the present invention employs the following aspects.
(1) An information processing device according to an aspect of the present invention includes an acquisition unit configured to acquire a surrounding image of a vehicle and a face image of a driver of the vehicle, a risk detection unit configured to detect a risk object in the surrounding image, a visual recognition determination unit configured to determine whether the driver visually recognizes the risk object, on the basis of information relating to a position of the risk object and information relating to the face image, an abnormality determination unit configured to determine whether a first abnormality occurs in the driver, on the basis of whether a risk avoidance action to reduce a risk degree indicating a degree of risk of collision between the vehicle and the risk object is detected after determination is made that the driver visually recognizes the risk object, and a notification control unit configured to cause a notification unit to perform a first notification when determination is made that the first abnormality occurs in the driver.
(2) In the aspect of (1) described above, the information processing device may further include an alarm control unit configured to cause an alarm unit to perform an alarm when the risk avoidance action is not detected after determination is made that the driver visually recognizes the risk object.
(3) In the aspect of (2) described above, even when the risk avoidance action is not detected after determination is made that the driver visually recognizes the risk object, subsequently, when a change of a direction of a face and/or a direction of a visual line of the driver is detected from the face image and the risk avoidance action is detected, the alarm control unit may not cause the alarm unit to perform the alarm.
(4) In the aspect of any one of (1) to (3) described above, when a change of a direction of a face and/or a direction of a visual line of the driver equal to or greater than a predetermined threshold is detected from the face image, the abnormality determination unit may be configured to determine that a second abnormality occurs in the driver, and the notification control unit may be configured to cause the notification unit to perform a second notification when determination is made that the second abnormality occurs in the driver.
(5) In the aspect of any one of (1) to (4) described above, the abnormality determination unit may be configured to determine that the first abnormality occurs in the driver when an event that the risk avoidance action by the driver is not detected after determination is made that the driver visually recognizes the risk object occurs a predetermined number of times in a predetermined period.
(6) In the aspect of any one of (1) to (5) described above, the notification control unit may be configured to cause the notification unit to perform the first notification at a timing other than when the driver is driving the vehicle.
(7) An information processing system according to an aspect of the present invention includes the information processing device of the aspect of any one of (1) to (6) described above, a first terminal device that is used by the driver and is the notification unit, and a second terminal device that is used in a medical institution, in which the first notification includes a content regarding a recommendation to receive a medical examination of the medical institution, and the first terminal device causes the second terminal device to perform a notification on the basis of information indicating an intention to accept to receive the medical examination input by the driver.
(8) An information processing system according to an aspect of the present invention includes the information processing device of the aspect of any one of (1) to (6) described above, a first terminal device that is used by the driver and is not the notification unit in which the first notification is controlled by the notification control unit, and a second terminal device that is used by a user different from the driver and is the notification unit in which the first notification is controlled by the notification control unit, in which the second terminal device causes the first terminal device to perform a notification on the basis of information input by an operation of the user.
(9) An information processing method according to an aspect of the present invention causes a computer to execute processing of acquiring a surrounding image of a vehicle and a face image of a driver of the vehicle, processing of detecting a risk object in the surrounding image, processing of determining whether the driver visually recognizes the risk object, on the basis of information relating to a position of the risk object and information relating to the face image, processing of determining whether a first abnormality occurs in the driver, on the basis of whether a risk avoidance action to reduce a risk degree indicating a degree of risk of collision between the vehicle and the risk object is detected after determination is made that the driver visually recognizes the risk object, and processing of causing a notification unit to perform a first notification when determination is made that the first abnormality occurs in the driver.
(10) A non-transitory computer-readable storage medium according to an aspect of the present invention stores a program causing a computer to execute processing of acquiring a surrounding image of a vehicle and a face image of a driver of the vehicle, processing of detecting a risk object in the surrounding image, processing of determining whether the driver visually recognizes the risk object, on the basis of information relating to a position of the risk object and information relating to the face image, processing of determining whether a first abnormality occurs in the driver, on the basis of whether a risk avoidance action to reduce a risk degree indicating a degree of risk of collision between the vehicle and the risk object is detected after determination is made that the driver visually recognizes the risk object, and processing of causing a notification unit to perform a first notification when determination is made that the first abnormality occurs in the driver.

According to the aspects of the present invention, it is possible to detect dementia of the driver.

### BRIEF DESCRIPTION OF THE DRAWINGS

FIG. 1 is a diagram showing an example of a configuration and a usage environment of a driving monitoring device including an information processing device according to a first embodiment.

FIG. 2 is a diagram showing an example of the driving monitoring device.

FIG. 3 is a diagram showing an example of an image acquired by the driving monitoring device.

FIG. 4 is a diagram illustrating overlooking alarm processing that is performed by the driving monitoring device.

FIG. 5 is a diagram showing an example of information that is detected by a detection unit.

FIG. 6 is a diagram showing an example of an overlooking alarm that is performed by an alarm unit.

FIG. 7 is a diagram illustrating special alarm processing that is performed by the driving monitoring device.

FIG. 8 is a flowchart illustrating an example of a flow of processing that is executed by a necessity determination unit.

FIG. 9 is a diagram illustrating abnormality notification processing that is performed by the driving monitoring device.

FIG. 10 is a diagram showing an example of history information.

FIG. 11 is a flowchart illustrating an example of a flow of processing that is executed by an abnormality detection unit.

FIG. 12 is a diagram illustrating abnormality notification processing in an information processing system according to a second embodiment.

### DETAILED DESCRIPTION OF THE INVENTION

Hereinafter, an embodiment of an information processing device, an information processing system, an information processing method, and a storage medium of the present invention will be described with reference to the drawings. In the following description, a front direction of a vehicle is referred to as a plus X direction, a rear direction of the vehicle is referred to as a minus X direction, a right direction that is a width direction of the vehicle with respect to the plus X direction is referred to as a plus Y direction, a left direction is referred to as a minus Y direction, and a height direction of the vehicle that is a direction perpendicular to the X direction and the Y direction is referred to as a plus Z direction.

### First Embodiment

### Overall Configuration

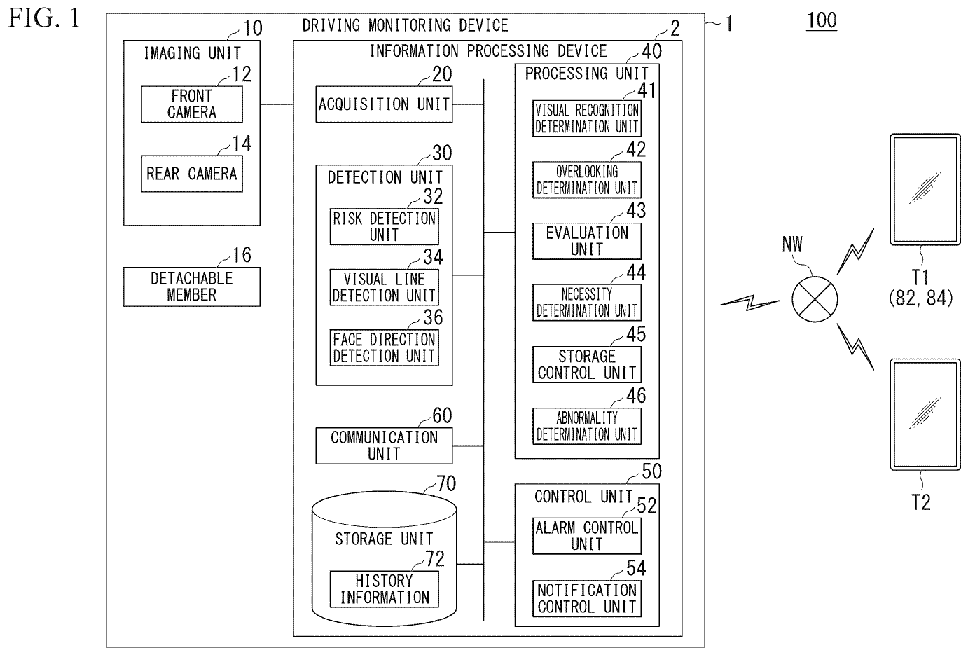

FIG. 1 is a diagram showing an example of a configuration and a usage environment of a driving monitoring device 1 including an information processing device 2 according to a first embodiment. The information processing device 2 is mounted in, for example, the driving monitoring device 1 that is retrofitted to a vehicle (hereinafter, referred to as a “vehicle M”) such as a drive recorder. That is, the driving monitoring device 1 (information processing device 2) is attachable and detachable to and from the vehicle M via a detachable member (hereinafter, referred to as a “detachable member 16”). The information processing device 2 is, for example, a device that is not connected to an in-vehicle network of the vehicle M. The in-vehicle network is an in-vehicle network using a communication standard such as a controller area network (CAN) to which an electronic control unit (ECU) regarding starting, braking, steering of the vehicle M, or vehicle control such as various sensors of the vehicle, operation members regarding driving, and operation buttons is connected. A network of the vehicle M for a human machine interface (HMI) may be excluded from the in-vehicle network.

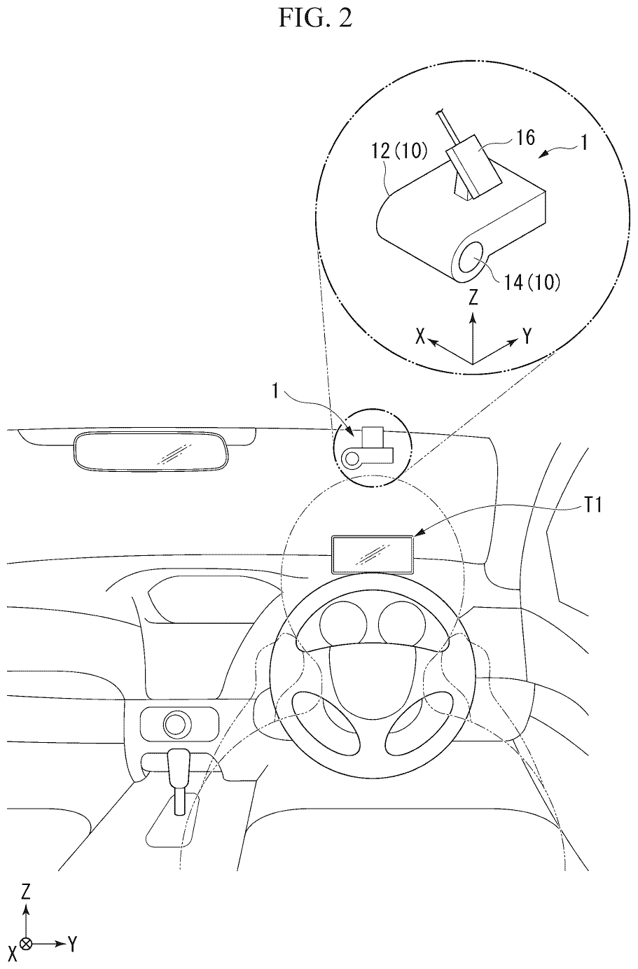

FIG. 2 is a diagram showing an example of the driving monitoring device 1. The driving monitoring device 1 is attached to, for example, a position in the vicinity of a front windshield of the vehicle M and near the front of a driver's seat. The driving monitoring device 1 stores an image (video) captured by the imaging unit 10 in, for example, a storage unit 70 (described below). The driving monitoring device 1 includes, for example, a display device such as a liquid crystal display (LCD), and may be configured to display an image (video) stored in the storage unit 70 on the display device.

The driving monitoring device 1 includes, for example, an imaging unit 10, the detachable member 16, and the information processing device 2. In the example shown in the drawing, the imaging unit 10 includes a front camera 12 and a rear camera 14.

Each of the front camera 12 and the rear camera 14 is, for example, a digital camera using a solid-state imaging element such as a charge coupled device (CCD) or a complementary metal oxide semiconductor (CMOS). The front camera 12 repeatedly (periodically) images the surroundings in front of the vehicle M from a position where the driving monitoring device 1 is attached. The rear camera 14 repeatedly (periodically) images the surroundings behind the vehicle M and the inside of a vehicle cabin of the vehicle M from the position where the driving monitoring device 1 is attached. An angle of view in a horizontal direction at which each of the front camera 12 and the rear camera 14 images is, for example, 180° or more. That is, the driving monitoring device 1 (imaging unit 10) images a range of 360° surrounding the vehicle M from the attached position. Each of the front camera 12 and the rear camera 14 may be, for example, a fisheye camera including a fisheye lens. Alternatively, each of the front camera 12 and the rear camera 14 may be a wide angle camera or the like. In the present embodiment, while a case where the front camera 12 and the rear camera 14 are used will be described, other cameras may be used instead of or in addition to the front camera 12 and the rear camera 14. That is, the imaging unit 10 may be configured such that an image (a surrounding image and a face image described below) for use in the present embodiment is imaged.

The detachable member 16 is, for example, a member for mounting the driving monitoring device 1 on the vehicle M. The detachable member 16 is any member, for example, a support member such as a sucker, a seal, or a bracket. The detachable member 16 may include a power supply cable (wire or the like) for supplying power to the driving monitoring device 1.

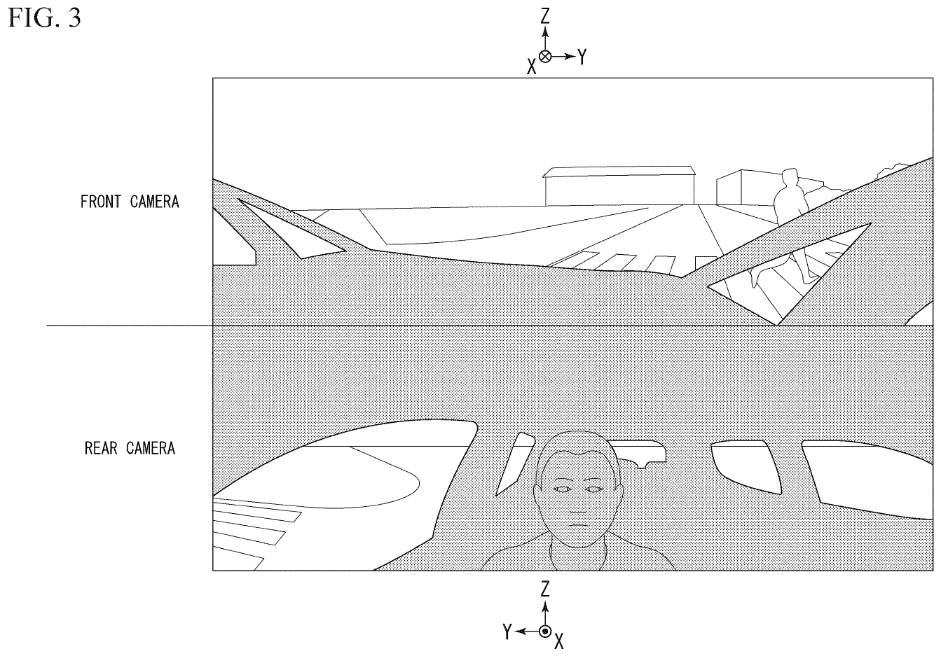

FIG. 3 is a diagram showing an example of an image acquired by the driving monitoring device 1. That is, FIG. 3 is a diagram showing an example of an image captured by the imaging unit 10. The front camera 12 images the front view of the vehicle M over a front windshield, a window on a driver's seat side, and a window on an assistant driver's seat side. The rear camera 14 images the inside (an occupant including a driver) of the vehicle cabin of the vehicle M, the rear view of the vehicle M over the window on the driver's seat side, the window on the assistant driver's seat, and a rear windshield, and the like.

In an image (hereinafter, referred to as a “front image”) captured by the front camera 12, as a subject, for example, the front view of the vehicle M seen through the front windshield such as another vehicle, a pedestrian, a bicycle, a fixed object, a road marking line and an object present on the right and left (front) of the vehicle M seen through the right and left windows are reflected.

In an image (hereinafter, referred to as a “rear image”) captured by the rear camera 14, the inside of the vehicle cabin of the vehicle M, the rear view of the vehicle M seen through the rear windshield, and an object present on the right and left (rear) of the vehicle M seen through the right and left windows are reflected. For this reason, in a rear view captured by the rear camera 14, as a subject, at least the driver of the vehicle M to which the driving monitoring device 1 is attached is reflected.

In the front image and the rear image, an image (hereinafter, referred to as a “surrounding image”) in which a surrounding situation of the vehicle M is imaged is included. In the rear image, an image (hereinafter, referred to as a “face image”) in which the face of the driver of the vehicle M is image is included. The driving monitoring device 1 may be attached on any place as long as the surrounding image and the face image can be captured by the imaging unit 10.

The information processing device 2 (see FIG. 1) outputs an alarm and notification via a first terminal device T1 on the basis of a result obtained by referring to the surrounding image and the face image output from the imaging unit 10. In the notification, the content regarding a recommendation to receive a medical examination of a medical institution may be included (details will be described later). The first terminal device T1 causes a second terminal device T2 to perform a notification on the basis of information indicating an intention to accept to receive the medical examination input by the driver of the vehicle M. The driving monitoring device 1, the first terminal device T1, and the second terminal device T2 may be collectively referred to as an “information processing system 100”.

The driving monitoring device 1, the first terminal device T1, and the second terminal device T2 are connected in a communicable manner via a network NW. The network W may be a network using wireless communication or may be a network using wired communication. The network W may be configured using, for example, the Internet or may be configured using a local area network (LAN), Bluetooth (Registered Trademark), or the like. The network W may be configured by a combination of a plurality of networks.

The first terminal device T1 is, for example, a portable terminal device such as a smartphone or a tablet terminal that is used by the driver who drives the vehicle M to which the driving monitoring device 1 is attached. In the first terminal device T1, for example, an application for receiving an alarm and notification from the information processing device 2 is executed. The application displays an image based on information (for example, alarm information and notification information described below) transmitted from the information processing device 2 on the display device or causes a speaker to emit sound. The first terminal device T1 is also referred to as an alarm unit 82 and a notification unit 84. The first terminal device T1 is used in a state of being detachably attached to the vehicle M, for example. For example, a holder for the first terminal device T1 having a detachable member is provided in one or both of the first terminal device T1 and the vehicle M, and the first terminal device T1 is supported by the holder.

A navigation, a display unit, or a speaker of the vehicle M may be used instead of the first terminal device T1. That is, the navigation, the display unit, or the speaker of the vehicle M may output an alarm or notification on the basis of an instruction of the information processing device 2. That is, the navigation of the vehicle M or the display unit or the speaker of the vehicle M may be the alarm unit 82 or the notification unit 84. A display unit (the alarm unit 82 and the notification unit 84), a speaker (the alarm unit 82 and the notification unit 84), or the like may be provided in the driving monitoring device 1 instead of the first terminal device T1.

The second terminal device T2 is a terminal device that is used in the medical institution. The second terminal device T2 may be a terminal that is used by personnel (for example, a physician or a nurse) belonging to the medical institution. The second terminal device T2 is an information processing device that is used by a user different from the driver who uses the first terminal device T1. The second terminal device T2 may be, for example, a smartphone, a tablet terminal, or a personal computer. In the second terminal device T2, for example, an application for receiving a notification by the first terminal device T1 is executed. The application may display an image based on information transmitted from the first terminal device T1 or may cause a speaker to emit sound.

As shown in FIG. 1, the information processing device 2 includes, for example, an acquisition unit 20, a detection unit 30, a processing unit 40, a control unit 50, a communication unit 60, and a storage unit 70. The detection unit 30 includes a risk detection unit 32, a visual line detection unit 34, and a face direction detection unit 36. The processing unit 40 includes a visual recognition determination unit 41, an overlooking determination unit 42, an evaluation unit 43, a necessity determination unit 44, a storage control unit 45, and an abnormality determination unit 46. The control unit 50 includes an alarm control unit 52 and a notification control unit 54.

For the acquisition unit 20, the detection unit 30, the processing unit 40, and the control unit 50, the function of each component is implemented, for example, by providing a hardware processor such as a central processing unit (CPU) and a storage device (a storage device including a non-transitory storage medium) storing a program (software) and causing the processor to execute the program. Some or all of these components may be implemented by hardware (including a circuit unit; circuitry) such as a large scale integration (LSI), an application specific integrated circuit (ASIC), a field-programmable gate array (FPGA), or a graphics processing unit (GPU) or the function of each component may be implemented by cooperation of software and hardware. For some or all of these components, the function of each component may be implemented by a dedicated LSI.

The program (software) may be stored in advance in the storage unit 70 (a storage device including a non-transitory storage medium) such as a read only memory (ROM), a random access memory (RAM), or a flash memory in the information processing device 2 or may be stored in a removable storage medium (a non-transitory storage medium) such as a memory card and installed on the storage device when the storage medium is loaded into the driving monitoring device 1. The program (software) may be downloaded from another computer device through short-range communication or wide area communication by the application executed on the first terminal device T1 and/or the second terminal device T2, transmitted from the first terminal device T1 and/or the second terminal device T2, and installed on the storage device.

In the storage unit 70, for example, history information 72, a program (for example, the program described above), and various other kinds of information are stored. The storage unit 70 may be implemented by various storage devices described above, an electrically erasable programmable read only memory (EEPROM), or the like. The communication unit 60 is an interface for performing wireless communication or wired communication with an information processing device (external device) such as the first terminal device T1 or the second terminal device T2. The communication unit 60 is an interface for performing wireless communication or wired communication with an information processing device (external device) such as the first terminal device T1 or the second terminal device T2 via the network W.

The driving monitoring device 1 (information processing device 2) performs at least overlooking alarm processing, special alarm processing, and abnormality notification processing. The overlooking alarm processing is processing of detecting overlooking of a risk object by the driver on the basis of the surrounding image and the face image and causing the alarm unit 82 to perform an alarm (hereinafter, referred to as an overlooking alarm). The special alarm processing is processing of causing the alarm unit 82 to perform an alarm (hereinafter, referred to as a special alarm) when the driver does not conduct a risk avoidance action although it is considered that the driver visually recognizes a risk object, on the basis of the surrounding image and the face image. The abnormality notification processing is processing of detecting an abnormality (for example, a disease such as dementia or visual field loss) of the driver on the basis of whether the driver conducts a risk avoidance action when it is considered that the driver visually recognizes a risk object, and causing the notification unit 84 to perform a notification.

The risk object is, for example, an object that the vehicle M should avoid. The risk object is, for example, an object with which the vehicle M interferes or is likely to interfere when moving in the present state. The object with which the vehicle M is likely to interfere is, for example, an object of which a degree of approach is equal to or greater than a predetermined degree after a predetermined time when a position, a moving direction, and a moving speed of an object and a position, a moving method, and a moving speed of the vehicle M are taken into consideration. The object may be a traffic participant such as a pedestrian, a bicycle, or a vehicle or may be an object such as an object placed on a road or a fallen object other than the traffic participant.

The risk avoidance action is an action to reduce a risk degree. The risk degree is an index indicating a degree of risk of collision between the vehicle M and the risk object. Examples of the risk avoidance action include a brake operation, a steering wheel operation toward an opposite side to the risk object.

### Overlooking Alarm Processing

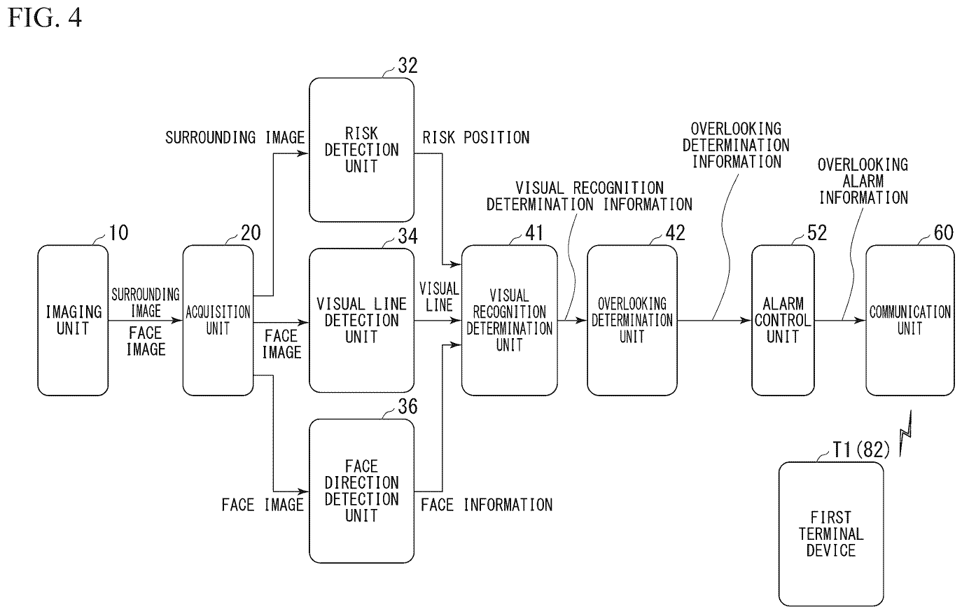

FIG. 4 is a diagram illustrating the overlooking alarm processing that is performed by the driving monitoring device 1 (information processing device 2).

The acquisition unit 20 acquires a front image and a rear image from the imaging unit 10. With this, the acquisition unit 20 acquires a surrounding image and a face image in association with each other. Because the imaging unit 10 repeatedly (periodically) captures the surrounding image and the face image, the acquisition unit 20 repeatedly (periodically) acquires the surrounding image and the face image. The acquisition unit 20 may acquire the surrounding image and the face image in association with a time (imaging time) at which these images are captured.

The risk detection unit 32 detects a risk object in the surrounding image. “Detecting a risk object” means, for example, detecting the presence of the risk object and a position (hereinafter, referred to as a risk position) of the risk object with respect to the vehicle M (driving monitoring device 1). For example, reference information (not shown) indicating a relationship between a risk position in the surrounding image and a relative position with respect to the vehicle M may be stored in advance in the storage unit 70. The risk detection unit 32 may detect the position of the risk object with respect to the vehicle M on the basis of the reference information.

The risk detection unit 32 detects a presence direction (approach direction) of a risk object with respect to the vehicle M by detecting the relative position of the risk object with respect to the vehicle M. “A presence direction of a risk object” means, for example, the presence direction of the risk object approaching the vehicle M (driving monitoring device 1) with respect to the vehicle M (driving monitoring device 1). Hereinafter, “the presence direction of the risk object” may be referred to as a “risk presence direction”.

The visual line detection unit 34 detects a visual line of the driver in the face image. Specifically, the visual line detection unit 34 detects the visual line of the driver by analyzing the face image using a predetermined image analysis method. “Detecting a visual line” means, for example, detecting a position of an eyeball of the driver and a direction of the visual line (a direction of the eyeball).

The face direction detection unit 36 detects at least a direction of a face of the driver in the face image. Specifically, the face direction detection unit 36 detects the direction of the face of the driver by analyzing the face image using a predetermined image analysis method. The face direction detection unit 36 may detect a position of the face of the driver in addition to the direction of the face of the driver. Hereinafter, information in which “the direction of the face” and “the position of the face” are combined may be referred to as “face information”. The face direction detection unit 36 detects the face information in the face image.

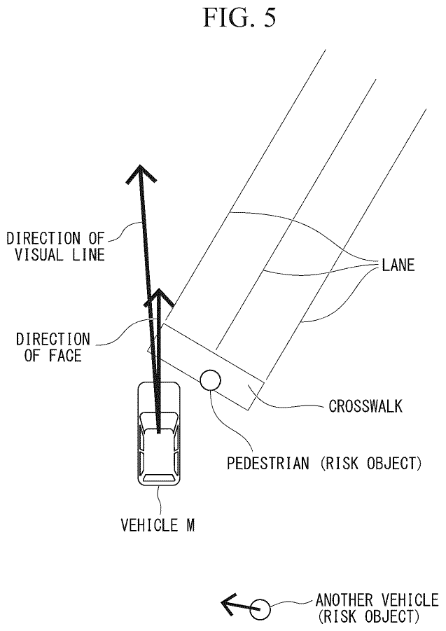

The detection unit 30 may be configured to detect a lane, a crosswalk, or the like in addition to the risk object, the visual line, and the face information described above. FIG. 5 is a diagram showing an example of information that is detected by the detection unit 30. In the example of FIG. 5, a pedestrian, another vehicle, lanes, and a crosstalk detected by the detection unit 30 are shown with the vehicle M, the direction of the visual line, and the direction of the face as a reference. The pedestrian and another vehicle are an example of a traffic participant (object) that is likely to be a risk object. For information that is detected by the detection unit 30, a traffic participant (object) that is detected by the detection unit 30 and is likely to be a risk object may be shown with the vehicle M, a direction of the vehicle M, and/or a proceeding direction of the vehicle M as a reference instead of the vehicle M, the direction of the visual line, and the direction of the face.

The visual recognition determination unit 41 (see FIG. 4) determines whether the driver visually recognizes the risk object, on the basis of the risk position and/or the risk presence direction, the visual line and/or the face information. Each of the “risk position” and the “risk presence direction” is an example of “information relating to a position of a risk object”. Each of the “visual line” and the “face information” is an example of “information relating to a face image”.

For example, reference information (not shown) indicating a relationship between a direction of an eyeball (visual line) and/or a direction of a face of the driver in the face image and a relative direction with respect to the proceeding direction of the vehicle M may be stored in advance in the storage unit 70. The visual recognition determination unit 41 may determine whether the direction of the visual line and/or the direction of the face match the risk presence direction with reference to the reference information. When the direction of the visual line and/or the direction of the face match the risk presence direction, the visual recognition determination unit 41 may determine that the driver visually recognizes the risk object. “A case where the direction of the visual line and/or the direction of the face match the risk presence direction” may be, for example, a case where the risk position is present on a straight line along the direction of the visual line and/or the direction of the face. Alternatively, “a case where the direction of the visual line and/or the direction of the face match the risk presence direction” may be a case where the risk position is present in a conical area which has a center axis along the direction of the visual line and/or the direction of the face with the position of the visual point (eyeball) and/or the face as a vertex and in which the side spreads toward the front. When the direction of the visual line and/or the direction of the face do not match the position of the risk object, the visual recognition determination unit 41 may determine that the driver does not visually recognize the risk object.

The visual recognition determination unit 41 generates visual recognition determination information on the basis of the determination result. In the visual recognition determination information, for example, the presence or absence of the above-described visual recognition and the risk presence direction are included. It should be noted that a determination method of visual recognition in the visual recognition determination unit 41 is not limited to the above-described method, and can be suitably changed.

The overlooking determination unit 42 determines the presence or absence of overlooking of the risk object by the driver on the basis of the visual recognition determination information.

For example, the overlooking determination unit 42 may determine that the overlooking of the risk object by the driver is present when a state in which the driver does not visually recognize the risk object is continued for a predetermined time or more. The overlooking determination unit 42 may determine that the overlooking of the risk object by the driver is absent when the driver visually recognizes the risk object or when a state in which the driver does not visually recognize the risk object is resolved before the predetermined time elapses. It should be noted that a determination method of overlooking by the overlooking determination unit 42 is not limited to the above-described method, and can be suitably changed.

The overlooking determination unit 42 generates overlooking determination information on the basis of the determination result. In the overlooking determination information, for example, the presence or absence of overlooking and the risk presence direction described above are included.

The alarm control unit 52 causes the first terminal device T1 (alarm unit 82) to output an overlooking alarm on the basis of the overlooking determination information. For example, the alarm control unit 52 transmits overlooking alarm information to the first terminal device T1 via the communication unit 60. In the overlooking alarm information, for example, information indicating a necessity of the overlooking alarm and the risk presence direction are included. For example, when information indicating that “overlooking is present” is included in the overlooking determination information, the alarm control unit 52 generates the overlooking alarm information including information indicating that “the overlooking alarm is necessary”. For example, when information indicating that “overlooking is absent” is included in the overlooking determination information, the alarm control unit 52 generates the overlooking alarm information including information indicating that “the overlooking alarm is not necessary”. The first terminal device T1 outputs the overlooking alarm on the basis of the overlooking alarm information.

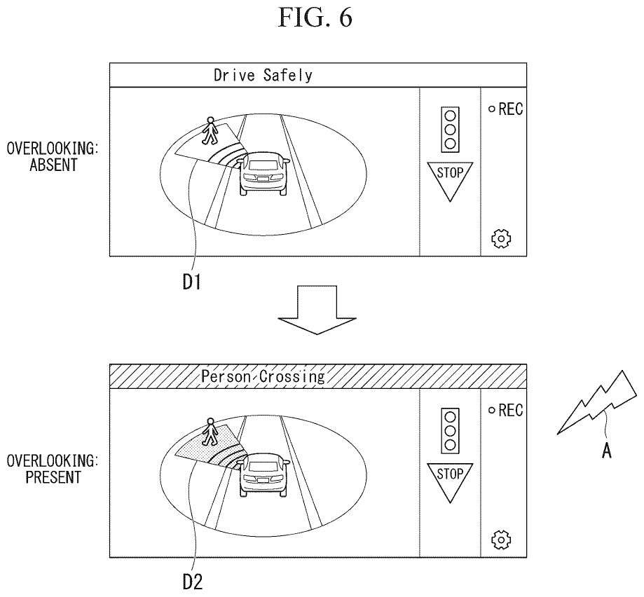

FIG. 6 is a diagram showing an example of the overlooking alarm that is performed by the first terminal device T1 (alarm unit 82). In the example of FIG. 6, the first terminal device T1 displays contents in which the risk presence direction (in the drawing, D1 and D2) can be recognized. Then, the first terminal device T1 performs the overlooking alarm by making an aspect of display of the risk presence direction different between when overlooking of the risk object is present (that is, when information indicating that “the overlooking alarm is necessary” is included in the overlooking alarm information) and when overlooking of the risk object is absent (that is, when information indicating that “the overlooking alarm is not necessary” is included in the overlooking alarm information). Specifically, when the overlooking of the risk object is present, the risk presence direction is displayed in a more emphasized aspect compared to when the overlooking is absent. The emphasized aspect may be, for example, an aspect that allows the driver to more easily visually recognize the risk presence direction such as emphasis by color or emphasis by blinking or turning on an icon or the like. The first terminal device T1 may perform the overlooking alarm through emission of sound by a speaker, light emission of a light, or the like in addition to or instead of the above-described method (in the drawing, A). A specific method of the overlooking alarm can be suitably changed as long as the overlooking alarm can be recognized by the driver.

The overlooking alarm by the first terminal device T1 (alarm unit 82) may be performed, for example, at a timing (that is, in real time) at which the driver is driving the vehicle M. Examples of “the timing at which the driver is driving the vehicle M” include a timing at which the vehicle M is traveling and a timing at which the vehicle M is temporarily stopped. For example, the transmission of the overlooking alarm information by the alarm control unit 52 may be performed at the above-described timing. Alternatively, the transmission of the overlooking alarm information by the alarm control unit 52 may be performed at any timing, and the first terminal device T1 (alarm unit 82) may control the timing of the overlooking alarm such that the overlooking alarm is performed at the above-described timing.

### Special Alarm Processing

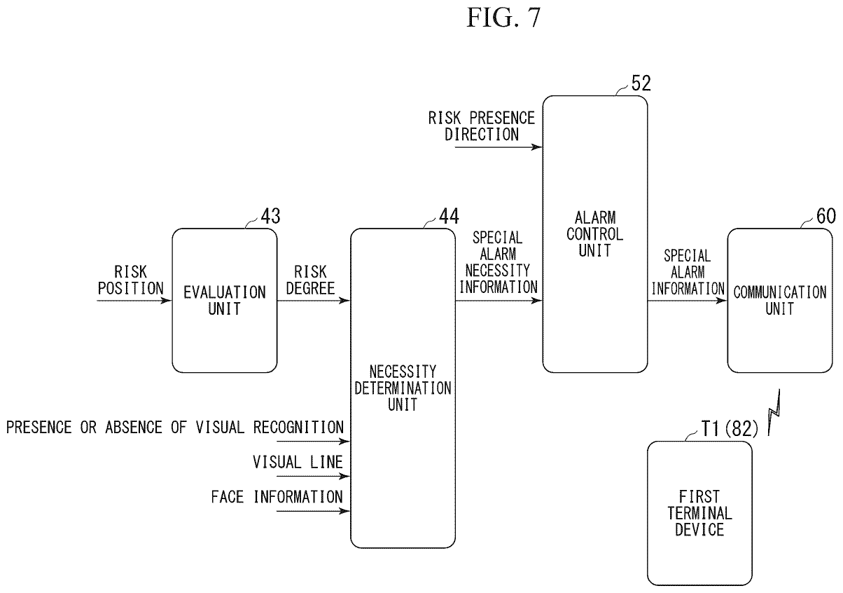

FIG. 7 is a diagram illustrating the special alarm processing that is performed by the driving monitoring device 1 (information processing device 2).

The evaluation unit 43 evaluates the risk degree on the basis of the risk position. The evaluation unit 43 may calculate a relative speed (hereinafter, referred to as a risk relative speed) of the risk object with respect to the vehicle M on the basis of a temporal change of the risk position detected by the risk detection unit 32, for example. Then, the evaluation unit 43 may evaluate the risk degree on the basis of the risk position (a relative position of the risk object with respect to the vehicle M) and the risk relative speed. For example, the evaluation unit 43 may calculate a possibility of collision of the risk object with the vehicle M within a predetermined time on the basis of the risk position and the risk relative speed. Then, the evaluation unit 43 may evaluate the risk degree to be higher as the calculated possibility has a higher value, and may evaluate the risk degree to be lower as the calculated possibility has a lower value.

The necessity determination unit 44 determines a necessity of a special alarm on the basis of, for example, the risk degree, the presence or absence of visual recognition of the risk object by the driver, and the visual line and/or the face information. The presence or absence of the visual recognition is obtained through, for example, the processing by the visual recognition determination unit 41 in the above-described overlooking alarm processing (see FIG. 4). Similarly, the visual line and the face information are obtained through, for example, the processing by the visual line detection unit 34 and the face direction detection unit 36 in the overlooking alarm processing (see FIG. 4). The necessity determination unit 44 generates special alarm necessity information including a determination result of the necessity of the special alarm.

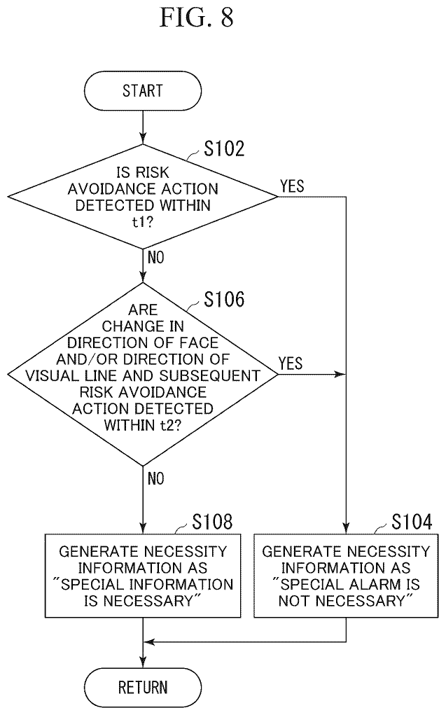

FIG. 8 is a flowchart illustrating an example of a flow of processing that is executed by the necessity determination unit 44. The processing shown in FIG. 8 is started each time the driver visually recognizes a risk object (that is, each time determination of visual recognition is made by the visual recognition determination unit 41).

First, the visual recognition determination unit 41 determines where the risk avoidance action by the driver is detected within a predetermined time (hereinafter, denoted as t1) after the driver visually recognizes the risk object (Step S102). t1 is, for example, a value determined in advance. t1 may be determined on the basis of a time normally required until the driver having a normal cognitive ability (for example, is not suffering from dementia or visual field loss) conducts the risk avoidance action after visually recognizing the risk object. The necessity determination unit 44 may detect the risk avoidance action on the basis of, for example, the risk degree evaluated by the evaluation unit 43. The necessity determination unit 44 may consider that there is the risk avoidance action, for example, when the risk degree evaluated by the evaluation unit 43 is reduced, and may detect the risk avoidance action accordingly. When the risk avoidance action is detected within t1 (Step S102; YES), the necessity determination unit 44 generates special alarm necessity information as “a special alarm is not necessary” (Step S104). Thereafter, the necessity determination unit 44 ends the processing of the flowchart shown in FIG. 8.

When the risk avoidance action is not detected within t1 (Step S102; NO), the necessity determination unit 44 determines whether a change of the direction of the face and/or the direction of the visual line of the driver and a subsequent risk avoidance action are detected within a predetermined time (hereinafter, denoted as t2) after the driver visually recognizes the risk object (Step S106). t2 is set to a value longer than t1. t2 is, for example, a value determined in advance. When the change of the direction of the face and/or the direction of the visual line, and the subsequent risk avoidance action are detected within t2 (Step S106; YES), the necessity determination unit 44 generates the special alarm necessity information as “a special alarm is not necessary” (Step S104). Thereafter, the necessity determination unit 44 ends the processing of the flowchart shown in FIG. 8.

When the change of the direction of the face and/or the direction of the visual line is not detected within t2 or when the subsequent risk avoidance action is not detected even when the change of the direction of the face and/or the direction of the visual line is detected (Step S106; NO), the necessity determination unit 44 generates special alarm necessity information as “a special alarm is necessary” (Step S108). Thereafter, the necessity determination unit 44 ends the processing of the flowchart shown in FIG. 8.

According to the above-described processing, when the risk avoidance action by the driver is not detected after determination is made that the driver visually recognizes the risk object, determination is made that a special alarm is necessary. When the driver is suffering from dementia, an event that the driver is affected by reduction in cognitive ability due to dementia not to conduct a risk avoidance action in spite of visually recognizing the risk object may occur. By detecting such an event and informing the driver of the event in the form of a special alarm, an accident (collision between the risk object and the vehicle M, or the like) caused by not conducting the risk avoidance action can be prevented.

On the other hand, when the driver is suffering from visual field loss (for example, visual field narrowing), in a case where a risk object is positioned at a site (hereinafter, referred to as a loss site) where the visual field is lost, the driver cannot cognize the risk object. That is, even when the direction of the visual line or the direction of the face matches the risk presence direction and the driver seems to be able to visually recognize the risk object, there is a possibility that the driver cannot cognize the risk object. In such a case, when the direction of the face and/or the direction of the visual line of the driver are changed, a phenomenon that the risk object is out of the loss site and the driver can cognize the risk object that cannot be cognized may occur. Considering this phenomenon, in the above-described processing, even when the risk avoidance action by the driver is not detected after determination is made that the driver visually recognizes the risk object, subsequently, when a change of the direction of the face and/or the direction of the visual line of the driver is detected from the face image and a risk avoidance action is detected, determination is made that a special alarm is not necessary. With this, it is possible to more accurately determine the necessity of the special alarm.

Returning to FIG. 7, the alarm control unit 52 causes the first terminal device T1 (alarm unit 82) to output the special alarm on the basis of the risk presence direction and the special alarm necessity information. The special alarm is an example of an “alarm”. The risk presence direction is obtained through, for example, the processing by the risk detection unit 32 in the above-described overlooking alarm processing (see FIG. 4). For example, the alarm control unit 52 transmits special alarm information to the first terminal device T1 via the communication unit 60. In the special alarm information, for example, the special alarm necessity information and the risk presence direction are included.

The first terminal device T1 outputs the special alarm on the basis of the special alarm information. The special alarm may be performed by displaying a character string, an image, or the like on, for example, the display device of the first terminal device T1 (alarm unit 82) on the basis of the special alarm information. For example, similarly to the above-described overlooking alarm, in contents in which the risk presence direction can be recognized, when the special alarm necessity information indicating that “the special alarm is necessary” is included in the special alarm information, the special alarm may be performed by displaying the risk presence direction in a more emphasized aspect. For example, in the special alarm, the risk presence direction may be displayed in a still more emphasized aspect than the above-described overlooking alarm. The first terminal device T1 may perform the special alarm through emission of sound by a speaker, light emission of a light, or the like in addition to or instead of the above-described method. Also in this case, the special alarm may be performed in a still more emphasized aspect than the overlooking alarm. For example, the special alarm may be performed through emission of sound at a greater volume level, blinking at a higher frequency, light emission at a stronger intensity, or the like than the overlooking alarm. A specific method of the special alarm can be suitably changed as long as the special alarm can be recognized by the driver.

The special alarm by the first terminal device T1 (alarm unit 82) may be performed, for example, at a timing (that is, in real time) at which the driver is driving the vehicle M. For example, the transmission of the special alarm information by the alarm control unit 52 may be performed at the above-described timing. Alternatively, the transmission of the special alarm information by the alarm control unit 52 may be performed at any timing, and the first terminal device T1 (alarm unit 82) may control the timing of the special alarm such that the special alarm is performed at the above-described timing.

### Abnormality Notification Processing

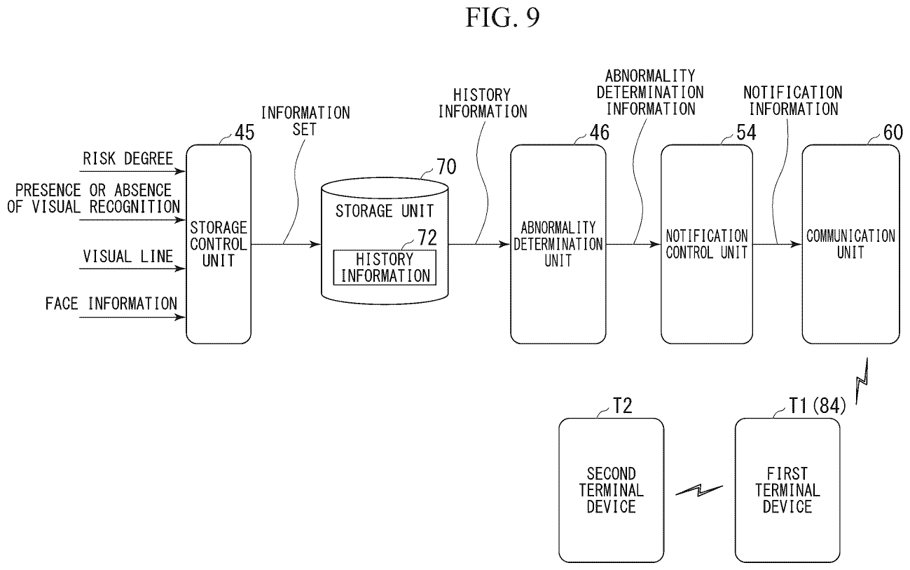

FIG. 9 is a diagram illustrating the abnormality notification processing that is performed by the driving monitoring device 1 (information processing device 2).

The storage control unit 45 stores a time, the risk degree, the presence or absence of the visual recognition of the risk object by the driver, and the visual line and/or the face information in the storage unit 70 in association with one another. The time corresponds, for example, the imaging time of the surrounding image and/or the face image used for the detection of the risk position, the determination of the presence or absence of the visual recognition, and the detection of the visual line or the face information. The risk degree is obtained through, for example, the processing by the evaluation unit 43 in the above-described special alarm processing (see FIG. 7). The presence or absence of the visual recognition is obtained through, for example, the processing by the visual recognition determination unit 41 in the above-described overlooking alarm processing (see FIG. 4). Similarly, the visual line and the face information are obtained through, for example, the processing by the visual line detection unit 34 and the face direction detection unit 36 in the overlooking alarm processing (see FIG. 4).

The above-described processing (storage processing) by the storage control unit 45 is performed, for example, each time the evaluation of the risk degree, the determination of the presence or absence of the visual recognition, and the detection of the visual line and/or the face information are performed. Hereinafter, information that is stored in the storage unit 70 in this manner is referred to as history information 72.

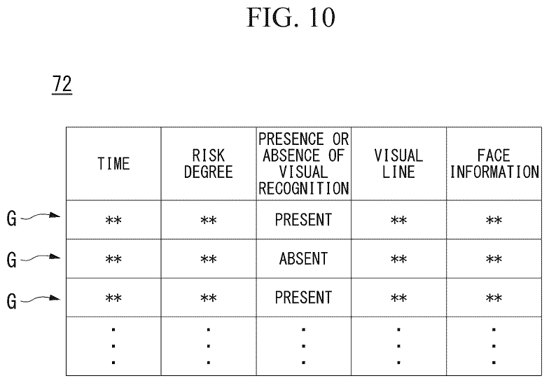

FIG. 10 is a diagram showing an example of the history information 72. The above-described storage processing is repeated by the storage control unit 45, so that a plurality of sets (hereinafter, referred to as an information set G) of information including the time, the risk degree, the presence or absence of the visual recognition, and the visual line and/or the face information are accumulated in the history information 72. That is, the storage control unit 45 generates the information set G each time the evaluation of the risk degree, the determination of the presence or absence of the visual recognition, and the detection of the visual line and/or the face information are performed. Then, the storage control unit 45 updates the history information 72 by adding the generated information set G to the history information 72.

Returning to FIG. 9, the abnormality determination unit 46 determines whether an abnormality occurs in the driver, on the basis of the history information 72. Specifically, the abnormality determination unit 46 determines whether a first abnormality occurs in the driver and whether a second abnormality occurs in the driver. The first abnormality is, for example, that the driver is suffering from or is likely to be suffering from dementia. The second abnormality is, for example, that the driver is suffering from or is likely to be suffering from visual field loss such as visual field narrowing. The abnormality determination unit 46 generates abnormality determination information on the basis of a determination result. In the abnormality determination information, for example, information indicating the presence or absence of the first abnormality and information indicating the presence or absence of the second abnormality described above are included.

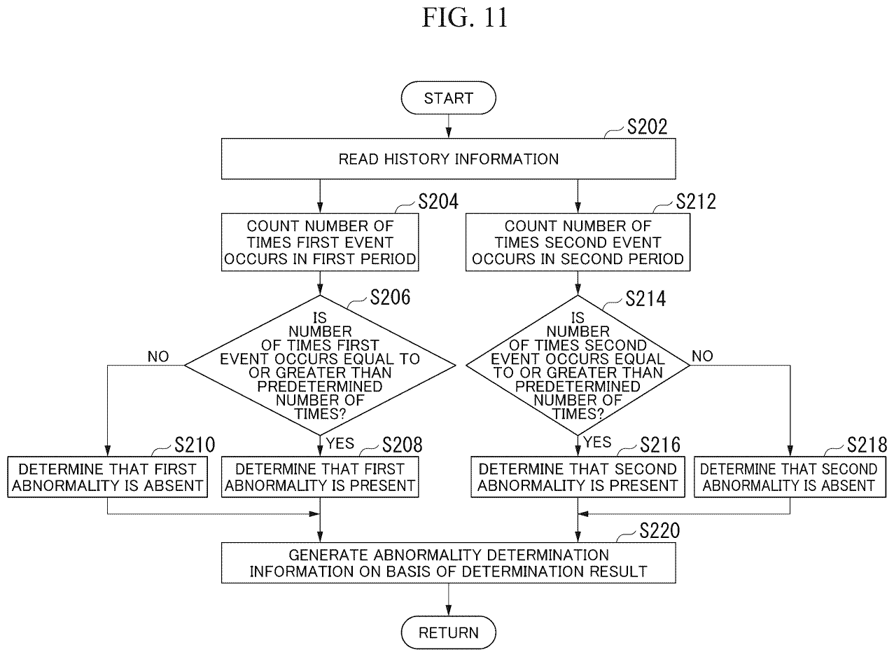

FIG. 11 is a flowchart illustrating an example of a flow of processing that is executed by the abnormality determination unit 46. The processing of the flowchart shown in FIG. 11 may be started, for example, repeatedly in a predetermined cycle, may be started each time the update of the history information 72 by the storage control unit 45 is performed, or may be started by an instruction of the driver or the like.

First, the abnormality determination unit 46 reads the history information 72 from the storage unit 70 (Step S202). After the processing of Step S202 is performed, the abnormality determination unit 46 executes processing of Steps S204 to S210 and processing of Steps S212 to S218 in parallel. It should be noted that the abnormality determination unit 46 may sequentially perform the processing of Steps S204 to S210 and the processing of Steps S212 to S218. The processing of Steps S204 to S210 is processing relating to the first abnormality. The processing of Steps S212 to Step S218 is processing relating to the second abnormality.

In processing of Step S204, the abnormality determination unit 46 counts the number of times a first event occurs in a first period, on the basis of the read history information 72. The first period is a period determined in advance. For example, the first period may be a period from a certain past point determined in advance to the present time.

The first event is, for example, an event satisfying both conditions (1-1) and (1-2) described below.
(1-1) “the risk avoidance action is not detected within t3 after determination is made that the driver visually recognizes the risk object”
(1-2) “the change of the direction of the face and/or the direction of the visual line is not detected within t4 after determination is made that the driver visually recognizes the risk object” or “when when the change of the direction of the face and/or the direction of the visual line is detected within t4 after determination is made that the driver visually recognizes the risk object, subsequently, the risk avoidance action is not detected”

Here, t3 is a predetermined time. t3 is, for example, a value determined in advance. t3 may be determined on the basis of a time normally required until the driver having a normal cognitive ability (for example, is not suffering from dementia or visual field loss) conducts the risk avoidance action after visually recognizing the risk object. t3 may be, for example, the same value as t1 described above. t4 is a predetermined time. t4 is set to a value longer than t3.t4 is, for example, a value determined in advance. t4 may be, for example, the same value as t2 described above.

The abnormality determination unit 46 can detect an event satisfying the above-described condition (1-1) by referring to information on the time, information on the risk degree, and information on the presence or absence of the visual recognition included in the history information 72, for example. The abnormality determination unit 46 can detect an event satisfying the above-described condition (1-2) by referring to information on the time, information on the risk degree, information on the presence or absence of the visual recognition, and information on the direction of the face and/or information on the direction of the visual line included in the history information 72. Similarly to the necessity determination unit 44, the abnormality determination unit 46 may consider that there is the risk avoidance action when the risk degree is reduced, and may detect the risk avoidance action accordingly.

Next, the abnormality determination unit 46 determines whether the number of times the first event occurs in the first period is equal or greater than a predetermined number of times (Step S206). The predetermined number of times may be, for example, one time or may be multiple times. When the number of times the first event occurs is equal or greater than the predetermined number of times (Step S206; YES), the abnormality determination unit 46 determines that there is the first abnormality in the driver (Step S208). When the number of times the first event occurs is not equal or greater than the predetermined number of times (Step S206; NO), the abnormality determination unit 46 determines that there is no first abnormality in the driver (Step S210).

According to the processing of Steps S204 to S210 described above, determination is performed about whether the first abnormality occurs in the driver, on the basis of whether the risk avoidance action is detected after determination is made that the driver visually recognizes the risk object.

By setting the above-described predetermined number of times to multiple times, it is possible to increase the accuracy of the determination of the first abnormality. That is, even when an event (first event) that the driver does not take the risk avoidance action in spite of visually recognizing the risk object occurs only one time, it does not mean that the driver is necessarily suffering from dementia. For example, there is a possibility that the driver does not take a risk avoidance action accidently such as a case where the driver is driving carelessly on that day. By employing a configuration in which the first event occurs multiple times and the first abnormality is initially determined, it is possible to suppress erroneous determination due to accidental occurrence of the first event as described above, and to increase determination accuracy.

By providing the condition (1-2), even when the risk avoidance action is not detected after determination is made that the driver visually recognizes the risk object, subsequently, when the change of the direction of the face and/or the direction of the visual line of the driver is detected from the face image and the risk avoidance action is detected, determination is made that the first abnormality does not occur. As described above, when the driver is suffering from visual field loss, a phenomenon that the risk object cannot be temporarily cognized by a positional relationship between the loss site and the risk object, but the driver can cognize the risk object with the change of the direction of the face and/or the direction of the visual line may occur. By employing a configuration in which the first abnormality is not determined in such a case, it is possible to increase the determination accuracy of the first abnormality. As described below, in the above-described case, the abnormality determination unit 46 determines that there is the second abnormality (that is, an abnormality relating to visual field loss) in the driver.

In processing of Step S212, the abnormality determination unit 46 counts the number of times a second event occurs in a second period, on the basis of the read history information 72. The second period is a period determined in advance. For example, the second period may be a period from a certain past point to the present time. The second period may be the same period as or a different period from the above-described first period.

The second event is, for example, an event satisfying at least one of conditions (2-1) and (2-2) described below.
(2-1) “the direction of the direction of the face and/or the direction of the visual line, and the subsequent risk avoidance action are detected within t4 after determination is made that the driver visually recognizes the risk object”
(2-2) “the direction of the face and/or the direction of the visual line of the driver are changed by a predetermined threshold or more”

The abnormality determination unit 46 can detect an event satisfying the above-described condition (2-1) by referring to information on the time, information on the risk degree, and information on the presence or absence of the visual recognition included in the history information 72, for example. The abnormality determination unit 46 can detect an event satisfying the above-described condition (2-2) by referring to information on the direction of the face and/or information on the direction of the visual line, for example. “The direction of the face and/or the direction of the visual line are changed by the predetermined threshold or more” means, for example, that the direction of the face and/or the direction of the visual line are changed at a predetermined angle or more within a predetermined time.

Next, the abnormality determination unit 46 determines whether the number of times the second event occurs in the second period is equal to or greater than a predetermined number of times (Step S214). The predetermined number of times may be, for example, one time or may be multiple times. The predetermined number of times in Step S214 may be the same as or different from the predetermined number of times in Step S206 described above. When the number of times the second event occurs is equal to or greater than the predetermined number of times (Step S214; YES), the abnormality determination unit 46 determines that there is the second abnormality in the driver (Step S216). When the number of times the second event occurs is not equal to or greater than the predetermined number of times (Step S214; NO), the abnormality determination unit 46 determines that there is no second abnormality in the driver (Step S218).

According to the processing of Steps S212 to S218 described above, when the change of the direction of the face and/or the direction of the visual line of the driver is detected from the face image, and subsequently, the risk avoidance action is detected, determination is made that there is the second abnormality in the driver. In addition to this determination, even when the direction of the face and/or the direction of the visual line of the driver are changed by the predetermined threshold or more is detected from the face image, determination is made that there is the second abnormality in the driver. When the driver is aware of visual field loss, or the like, the driver may drive the vehicle M while actively changing the direction of the face and/or the direction of the visual line. By providing the condition (2-2), it is also possible to detect visual field loss based on such an action of the driver.

After the processing of Steps S204 to S210 and the processing of Steps S212 to S218 end, the abnormality determination unit 46 generates the abnormality determination information on the basis of the above-described determination results. That is, the abnormality determination unit 46 generates the abnormality determination information on the basis of the determination result relating to the presence or absence of the first abnormality and the determination result relating to the presence or absence of the second abnormality.

Returning to FIG. 9, the notification control unit 54 causes the first terminal device T1 (notification unit 84) to output a notification on the basis of the abnormality determination information. For example, the notification control unit 54 transmits notification information to the first terminal device T1 (notification unit 84) via the communication unit 60. For example, the notification control unit 54 may transmit the notification information to the first terminal device T1 when at least one of information indicating that there is the first abnormality in the driver and information indicating that there is the second abnormality in the driver is included in the abnormality determination information (that is, when the abnormality determination unit 46 determines that at least one of the first abnormality and the second abnormality occurs in the driver). In the notification information, for example, information indicating the content (first abnormality, second abnormality) of the abnormality of the driver is included. The notification control unit 54 may not transmit the notification information to the first terminal device T1 when both information indicating that there is no first abnormality in the driver and information indicating that there is no second abnormality in the driver are included in the abnormality determination information (that is, when the abnormality determination unit 46 determines that none of the first abnormality and the second abnormality occurs in the driver).

The first terminal device T1 (notification unit 84) outputs a notification on the basis of the notification information. The notification may be performed, for example, by displaying a character string, an image, or the like based on the notification information on the display device of the first terminal device T1 (notification unit 84). The first terminal device T1 (notification unit 84) may perform a notification according to the content (first abnormality, second abnormality) of the abnormality of the driver. For example, when information indicating the first abnormality is included in the notification information, the first terminal device T1 (notification unit 84) may perform a notification indicating that the driver is suffering from or is likely to be suffering from dementia. This notification is an example of a “first notification”. When information indicating the second abnormality is included in the notification information, the first terminal device T1 (notification unit 84) may perform a notification indicating that the driver is suffering from or is likely to be suffering from visual field loss. This notification is an example of a “second notification”.

In the notification (the first notification and the second notification) by the first terminal device T1 (notification unit 84), the content regarding a recommendation to accept to receive a medical examination of a medical institution (for example, a family doctor of the driver) may be included. In this case, the first terminal device T1 (notification unit 84) may cause the driver to input information indicating whether the driver accepts to receive the medical examination of the medical institution. Specifically, a virtual button or the like indicating that the driver accepts to receive the medical examination may be displayed on the display device of the first terminal device T1 (notification unit 84). Such a button or the like functions as an acceptance information acquisition unit that acquires information (hereinafter, referred to as acceptance information) indicating the intention of the driver to accept to receive the medical examination of the medical institution. Then, when the driver operates the first terminal device T1 (notification unit 84), and the acceptance information acquisition unit acquires the acceptance information, the first terminal device T1 (notification unit 84) may cause the second terminal device T2 to perform a notification indicating that the driver accepts to receive the medical examination.

It should be noted that an aspect of notification by the first terminal device T1 (notification unit 84) is not particularly limited as long as the driver can recognize the aspect, and can be suitably changed.

The notification by the first terminal device T1 (notification unit 84) may be performed, for example, at a timing other than the timing at which the driver is driving the vehicle M. Examples of “the timing other than the timing at which the driver is driving the vehicle M” include a timing at which the vehicle M is stopped, a timing after the drivers ends the driving of the vehicle M, and a timing at which the driver is separated from the vehicle M and stays at home. For example, the transmission of the notification information by the notification control unit 54 may be performed at the above-described timing. Alternatively, the transmission of the notification information by the notification control unit 54 may be performed at any timing, and the first terminal device T1 (notification unit 84) may control a timing of notification such that the notification is performed at the above-described timing.

According to the first embodiment described above, the information processing device 2 includes the acquisition unit 20 that acquires the surrounding image of the vehicle M and the face image of the driver of the vehicle M, the risk detection unit 32 that detects the risk object in the surrounding image, the visual recognition determination unit 41 that determines whether the driver visually recognizes the risk object, on the basis of information relating to the position of the risk object and information relating to the face image, the abnormality determination unit 46 that determines whether the first abnormality occurs in the driver, on the basis of whether the risk avoidance action to reduce the risk degree indicating the degree of risk of collision between the vehicle M and the risk object is detected after determination is made that the driver visually recognizes the risk object, and the notification control unit 54 that causes the notification unit 84 to perform the notification when determination is made that the first abnormality occurs in the driver.

### Second Embodiment

Next, while a second embodiment will be described, a basic configuration is similar to the first embodiment. For this reason, similar configurations are represented by the same reference numbers, and description thereof will not be repeated. Only differences will be described.

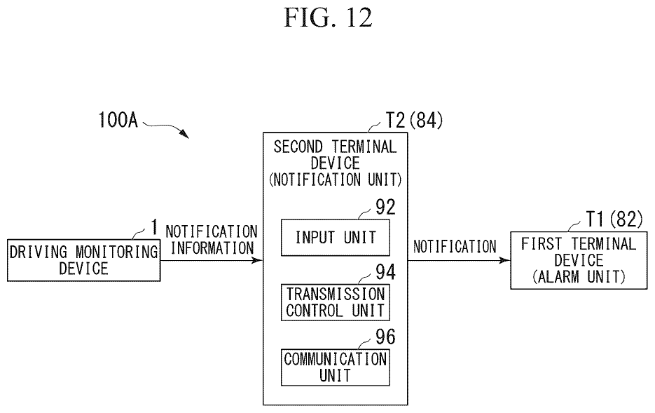

FIG. 12 is a diagram illustrating abnormality notification processing in an information processing system 100A according to the second embodiment. Similarly to the first embodiment, a second terminal device T2 in the second embodiment may be a terminal device that is used in the medical institution. Alternatively, the second terminal device T2 may be an information processing device that is used by a staff member of an insurance company.

In the second embodiment, the driving monitoring device 1 (notification control unit 54) transmits the notification information to the second terminal device T2, not to the first terminal device T1. That is, in the second embodiment, the notification unit 84 in which the notification (first notification, second notification) is controlled by the notification control unit 54 is the second terminal device T2, not the first terminal device T1. In the second terminal device T2, for example, an application for receiving the notification by the information processing device 2 is executed. The application displays an image, a character string, or the like based on information (notification information or the like) transmitted from the information processing device 2 on the display device or causes a speaker to emit sound.

The second terminal device T2 causes the first terminal device T1 to perform a notification on the basis of information input by an operation of the user. The second terminal device T2 includes, for example, an input unit 92, a transmission control unit 94, and a communication unit 96.

For the input unit 92 and the transmission control unit 94, the function of each component is implemented, for example, by providing a hardware processor such as a CPU and a storage device (a storage device including a non-transitory storage medium) storing a program (software) and causing the processor to execute the program. Some or all of these components may be implemented by hardware (including a circuit unit) such as an LSI, an ASIC, an FPGA, or a GPU or the function of each component may be implemented by cooperation of software and hardware. For some or all of these components, the function of each component may be implemented by a dedicated LSI. The communication unit 96 is an interface for performing wireless communication or wired communication with an information processing device such as the information processing device 2 or the first terminal device T1 via the network W.

The input unit 92 receives an operation of the user and inputs the operation to the second terminal device T2. The input unit 92 is configured using existing input devices such as a keyboard, a pointing device (a mouse, a tablet, or the like), a button, and a touch panel. The input unit 92 may be an interface for connecting an input device to the second terminal device T2. In this case, the input unit 92 inputs an input signal generated according to an operation of the user on the input device to the second terminal device T2. The input unit 92 may have any configuration as long as a configuration is made in which an instruction based on an operation of the user can be input to the second terminal device T2.

For example, the user confirms the content of the notification from the second terminal device T2 caused by the driving monitoring device 1 (notification control unit 54), and examines the content that should be notified to the driver. The content to be notified to the driver is, for example, a recommendation of a medical examination of a medical institution for finding out the possibility of a disease (abnormality) such as dementia or visual field loss or visual field loss. The user inputs information including the examined notification content to the second terminal device T2 by operating the input unit 92. The transmission control unit 94 transmits information input by the operation of the user to the first terminal device T1 via the communication unit 96. The first terminal device T1 performs a notification to the driver on the basis of the transmitted information.

The information processing system 100A may be configured to regulate the transmission of the notification information to the second terminal device T2 by the driving monitoring device 1 (notification control unit 54) according to an operation of the driver on the first terminal device T1 or the driving monitoring device 1. For example, regulation information indicating the presence or absence of regulation of the transmission of the notification information may be generated according to an operation of the driver on the first terminal device T1 or the driving monitoring device 1, and the generated regulation information may be stored in the storage unit 70. Then, the alarm control unit 52 may determine whether to transmit the notification information to the second terminal device T2, on the basis of the regulation information stored in the storage unit 70. According to such a configuration, determination regarding whether to inform the user who uses the second terminal device T2 of information relating to an abnormality of the driver is performed according to the intention of the driver. Accordingly, the privacy of the driver is easily protected.

According to the second embodiment described above, the information processing system 100A includes the information processing device 2, the first terminal device T1 that is used by the driver and is not the notification unit 84 in which the notification is controlled by the notification control unit 54, and the second terminal device T2 that is used by the user different from the driver and is the notification unit 84 in which the notification is controlled by the notification control unit 54, and the second terminal device T2 causes the first terminal device T1 to perform the notification on the basis of information input by the operation of the user. With this, the notification content examined by the user who uses the second terminal device T2 can be provided to the driver who uses the first terminal device T1.

### Modification Examples

The technical scope of the present invention is not limited to the above-described embodiments, and various changes may be made without departing from the spirit of the present invention.

For example, in the above-described embodiments, while the necessity determination unit 44 and the abnormality determination unit 46 detect the risk avoidance action on the basis of the reduction of the risk degree evaluated by the evaluation unit 43, a detection method of the risk avoidance action is not limited thereto. For example, an acceleration sensor may be provided in the driving monitoring device 1 or the first terminal device T1, and a risk avoidance action such as a brake operation or a steering wheel operation may be detected on the basis of information on an acceleration to be detected. Alternatively, various sensors provided in the vehicle M or an ECU of the vehicle M that manages these sensors may be connected to the network W, and the risk avoidance action may be detected on the basis of signals output from these sensors. A detection method of the risk avoidance action is not limited to these examples, and can be suitably changed.

In the above-described embodiments, while the abnormality determination unit 46 performs the determination of the presence or absence of the first abnormality and the determination of the presence or absence of the second abnormality on the basis of the history information 72 in which the time, the risk degree, the presence or absence of the visual recognition, and the visual line and/or the face information are associated with one another, a determination method of the abnormality is not limited thereto. For example, information indicating the results (for example, the result of the determination of Step S102 or S104) of various determinations in the special alarm processing (see FIG. 8) may be stored (accumulated) in the storage unit 70 for each determination. Then, the abnormality determination unit 46 may perform the determination of the abnormality on the basis of the above-described information stored (accumulated) in the storage unit 70. A determination method of the presence or absence of the abnormality is not limited to these examples, and can be suitably changed.

The driving monitoring device 1 (information processing device 2) may perform another alarm processing (hereinafter, referred to as auxiliary alarm processing) in addition to (or instead of) the above-described overlooking alarm processing. The auxiliary alarm processing may be, for example, collision alarm processing. The collision alarm processing may be processing of detecting a possibility (for example, the above-described risk degree) of collision of the risk object with the vehicle M on the basis of the position or relative speed of the risk object with respect to the vehicle M, and causing the alarm unit 82 to perform an alarm. In the collision alarm processing, the relative speed of the risk object with respect to the vehicle M may be calculated by analyzing a temporal change of the risk position detected by the risk detection unit 32 with the processing unit 40, for example. When the possibility of collision of the risk object with the vehicle M is detected by the processing unit 40 (for example, when the risk degree is equal to or greater than the predetermined threshold), the alarm control unit 52 may cause the alarm unit 82 to perform an alarm. Similarly to the overlooking alarm processing, the auxiliary alarm processing may be performed at a timing (that is, in real time) at which the driver is driving the vehicle M. In a configuration in which the driving monitoring device 1 (information processing device 2) performs the auxiliary alarm processing, the (overlooking alarm) based on the result of the determination of overlooking by the alarm overlooking determination unit 42 may not be performed. Also in this case, the determination of the presence or absence of the visual recognition by the visual recognition determination unit 41 is executed for the special alarm processing and the abnormality notification processing described above.

Information relating to the result of the determination of overlooking by the overlooking determination unit 42, and the like may be integrated in the storage unit 70, and contents (hereinafter, referred to as “review contents” that are generated on the basis of the integrated information and allows the driver to review his/her driving in the past may be provided to the driver by the first terminal device T1 (alarm unit 82). The review contents may include, for example, information (character string, image, or the like) that allows the driver to confirm a risk object overlooked by the driver. The review contents may be generated by the driving monitoring device 1 (information processing device 2) or may be generated by the first terminal device T1 (alarm unit 82).

In the information processing systems 100 and 100A according to the above-described embodiments, the driving monitoring device 1 (notification control unit 54) may transmit the notification information to both the first terminal device T1 and the second terminal device T2. In other words, both the first terminal device T1 and the second terminal device T2 may be the notification unit 84 in which the notification (first notification, second notification) is controlled by the notification control unit 54.

In the first embodiment, the driving monitoring device 1 (information processing device 2) and the first terminal device T1 are configured as different devices, but may be configured as an integrated device. For example, all the functions of the imaging unit 10, the acquisition unit 20, the detection unit 30, the processing unit 40, the control unit 50, the storage unit 70, the alarm unit 82, and the notification unit 84 may be mounted in the driving monitoring device 1. Alternatively, all these functions may be mounted in the first terminal device T1 or the vehicle M. Also in the second embodiment, similarly, all the functions of the imaging unit 10, the acquisition unit 20, the detection unit 30, the processing unit 40, the control unit 50, the storage unit 70, and the alarm unit 82 may be mounted in the driving monitoring device 1 or all these functions may be mounted in the first terminal device T1. A camera other than the camera in the driving monitoring device 1 may be allowed to have a part or all of the functions of the imaging unit 10. For example, a driver monitor camera that images the driver may capture the face image.

The driving monitoring device 1 may be mounted using a plurality of information processing devices. For example, the functions of the information processing device 2 may be mounted in the first terminal device T1 or the vehicle M (for example, the ECU). Alternatively, the driving monitoring device 1 may be mounted using a device such as a cloud. For example, in the driving monitoring device 1, the detection unit 30, the processing unit 40, the control unit 50, and the storage unit 70 may be mounted in different information processing devices. For example, the storage unit 70 may be distributed and mounted in a plurality of information processing devices.

The embodiments and the modification examples described above can be expressed as follows.

An information processing device including
a storage medium that stores computer-readable instructions, and
a processor connected to the storage medium,
in which the processor executes the computer-readable instructions to
acquire a surrounding image of a vehicle and a face image of a driver of the vehicle,
detect a risk object in the surrounding image,
determine whether the driver visually recognizes the risk object, on the basis of information relating to a position of the risk object and information relating to the face image,
determine whether a first abnormality occurs in the driver, on the basis of whether a risk avoidance action to reduce a risk degree indicating a degree of risk of collision between the vehicle and the risk object is detected after determination is made that the driver visually recognizes the risk object,
cause a notification unit to perform a first notification when determination is made that the first abnormality occurs in the driver.

The components in the embodiments described above can be appropriately replaced with well-known components without departing from the spirit of the present invention, and the embodiments and the modification examples described above may be appropriately combined.

What is claimed is:

1. An information processing device comprising:
an acquisition unit configured to acquire a surrounding image of a vehicle and a face image of a driver of the vehicle;
a risk detection unit configured to detect a risk object in the surrounding image;
a visual recognition determination unit configured to determine whether the driver visually recognizes the risk object, on the basis of information relating to a position of the risk object and information relating to the face image;
an abnormality determination unit configured to determine whether a first abnormality occurs in the driver, on the basis of whether a risk avoidance action to reduce a risk degree indicating a degree of risk of collision between the vehicle and the risk object is detected after determination is made that the driver visually recognizes the risk object; and
a notification control unit configured to cause a notification unit to perform a first notification when determination is made that the first abnormality occurs in the driver.

2. The information processing device according to claim 1, further comprising:
an alarm control unit configured to cause an alarm unit to perform an alarm when the risk avoidance action is not detected after determination is made that the driver visually recognizes the risk object.

3. The information processing device according to claim 2,
wherein even when the risk avoidance action is not detected after determination is made that the driver visually recognizes the risk object, subsequently, when a change of a direction of a face and/or a direction of a visual line of the driver is detected from the face image and the risk avoidance action is detected, the alarm control unit does not cause the alarm unit to perform the alarm.

4. The information processing device according to claim 1,
wherein, when a change of a direction of a face and/or a direction of a visual line of the driver equal to or greater than a predetermined threshold is detected from the face image, the abnormality determination unit is configured to determine that a second abnormality occurs in the driver, and
the notification control unit is configured to cause the notification unit to perform a second notification when determination is made that the second abnormality occurs in the driver.

5. The information processing device according to claim 1,
wherein the abnormality determination unit is configured to determine that the first abnormality occurs in the driver when an event that the risk avoidance action by the driver is not detected after determination is made that the driver visually recognizes the risk object occurs a predetermined number of times in a predetermined period.

6. The information processing device according to claim 1,
wherein the notification control unit is configured to cause the notification unit to perform the first notification at a timing other than when the driver is driving the vehicle.

7. An information processing system comprising:
the information processing device according to claim 1;
a first terminal device that is used by the driver and is the notification unit; and
a second terminal device that is used in a medical institution,
wherein the first notification includes content regarding a recommendation to accept to receive the medical institution, and
the first terminal device causes the second terminal device to perform a notification on the basis of information indicating an intention to accept to receive the medical examination input by the user.

8. An information processing system comprising:
the information processing device according to claim 1;
a first terminal device that is used by the driver and is not the notification unit in which the first notification is controlled by the notification control unit; and
a second terminal device that is used by a user different from the driver and is the notification unit in which the first notification is controlled by the notification control unit,
wherein the second terminal device causes the first terminal device to perform a notification on the basis of information input by an operation of the user.

9. An information processing method causing a computer to execute:
processing of acquiring a surrounding image of a vehicle and a face image of a driver of the vehicle;
processing of detecting a risk object in the surrounding image;
processing of determining whether the driver visually recognizes the risk object, on the basis of information relating to a position of the risk object and information relating to the face image;
processing of determining whether a first abnormality occurs in the driver, on the basis of whether a risk avoidance action to reduce a risk degree indicating a degree of risk of collision between the vehicle and the risk object is detected after determination is made that the driver visually recognizes the risk object; and
processing of causing a notification unit to perform a first notification when determination is made that the first abnormality occurs in the driver.

10. A non-transitory computer-readable storage medium storing a program causing a computer to execute:
processing of acquiring a surrounding image of a vehicle and a face image of a driver of the vehicle;
processing of detecting a risk object in the surrounding image;
processing of determining whether the driver visually recognizes the risk object, on the basis of information relating to a position of the risk object and information relating to the face image;
processing of determining whether a first abnormality occurs in the driver, on the basis of whether a risk avoidance action to reduce a risk degree indicating a degree of risk of collision between the vehicle and the risk object is detected after determination is made that the driver visually recognizes the risk object; and
processing of causing a notification unit to perform a first notification when determination is made that the first abnormality occurs in the driver.
